(19) United States
(12) Patent Application Publication
(10) Pub. No.: US 2017/0226282 A1
(43) Pub. Date: Aug. 10, 2017

(54) CHLOROPHYLL POLYMERS AND USES THEREOF

(71) Applicant: University of Calcutta, Kolkata (IN)

(72) Inventor: Nilanjan DEB, Kolkata (IN)

(73) Assignee: University of Calcutta, Kolkata (IN)

(21) Appl. No.: 15/424,292

(22) Filed: Feb. 3, 2017

(30) Foreign Application Priority Data
Feb. 4, 2016 (IN) 201631004055

(51) Int. Cl. C08G 61/12; C09B 67/08; G01N 21/78; G01N 33/00; G01N 27/12; C08L 65/00; G01N 33/02

(52) U.S. Cl. CPC C08G 61/124; C08L 65/00; C09B 67/0013; G01N 33/025; G01N 33/0047; G01N 27/126; G01N 21/783; C08G 2261/364; C08G 2261/94; C08G 2261/50; C08G 2261/792

(57) ABSTRACT

Provided herein are chlorophyll polymers and conductive materials, sensors, and devices comprising the chlorophyll polymers, and methods of use and preparation thereof.

### BACKGROUND

Fruits, vegetables, meats, fish, and milk are subjected to various supply chains that often require traveling long distances, temperature fluctuations, and handling by packaging and packing equipment. Such supply chains may result in the contamination by microorganisms such as bacteria and/or fungi, which can generate unpleasant odors and/or toxic substances that may be harmful to human and animal health. Volatile compounds have been identified that relate to the growth of several microorganisms in biological samples. Early detection of the microorganisms offers many advantages for quality control in the food industry. New sensor technologies are needed to identify high quality food including fruits, vegetables, meats, fish, and milk for producers and end-consumers.

### SUMMARY

The present technology relates to low cost chlorophyll polymers, which may be conductive, and may be used as sensors or devices for the rapid detection of food quality of fruits, vegetables, meats, fish, and milk. The sensors or devices may also be doped with various pigments that may be capable of determining food spoilage, e.g. detection of volatile organic compound(s) (“VOC”).

In one aspect the chlorophyll polymer may be a polymer of Formula A:

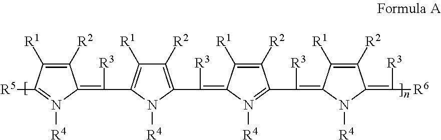

wherein R¹ at each occurrence may be independently hydrogen, C₁-C₆ alkyl, C₂-C₆ alkenyl, aldehyde, or a conductive polymer; R² at each occurrence may be independently hydrogen, C₁-C₆ alkyl, or aldehyde; R³ at each occurrence may be independently hydrogen or C₁-C₆ alkyl; R⁴ at each occurrence may be independently absent, hydrogen, or C₁-C₆ alkyl; R⁵ at each occurrence may be independently hydrogen or C₁-C₆ alkyl; R⁶ at each occurrence may be independently hydrogen or C₁-C₆ alkyl; and n may be an integer of from 2 to 2000. In some embodiments, the chlorophyll polymer may be Formula B:

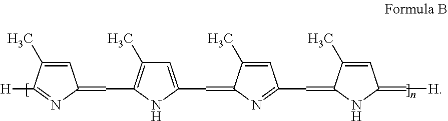

In one aspect, the chlorophyll polymer may be prepared by a process including heating a compound of Formulas I and/or II:

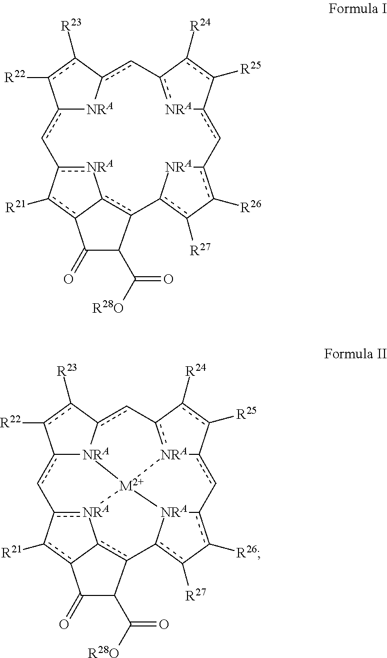

or a stereoisomer and/or tautomer thereof in a solution comprising R—CHO, wherein R may be C₁-C₄ alkyl; [symbol] represents a single bond or a double bond; R^{A} may be absent or a hydrogen; M²⁺ may be Mg²⁺, Fe²⁺, Co²⁺, Ni²⁺, or Cu²⁻; R²¹, R²², R²³, R²⁴, R²⁵, and R²⁶ at each occurrence may be independently hydrogen, C₁-C₄ alkyl, C₂-C₄ alkenyl, or aldehyde; R²⁷ may be a C₁-C₄ alkyl or C₂-C₄ alkenyl, wherein the C₁-C₄ alkyl or C₂-C₄ alkenyl may be optionally substituted with —C(O)OR²⁹, wherein R²⁹ may be hydrogen, C₁-C₂₄ alkyl, or C₂-C₂₄ alkenyl; and R²⁸ may be hydrogen or C₁-C₄ alkyl. In some embodiments, the compounds of Formulas I and II may be compounds of Formulas III and IV:

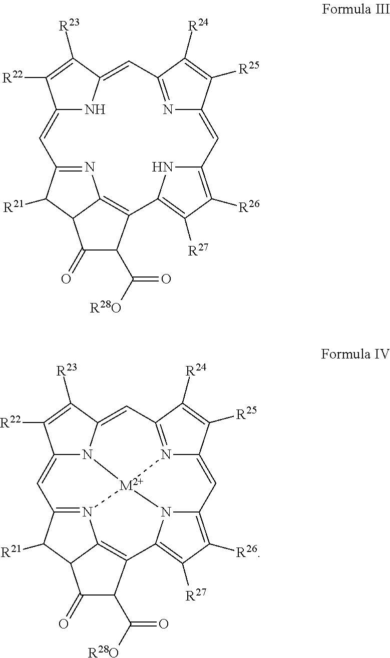

In some embodiments, the compound of Formula I may be a demetallized chlorophyll. In some embodiments, the compound of Formula II may be chlorophyll.

In another aspect, the present technology provides a compound of the formula:

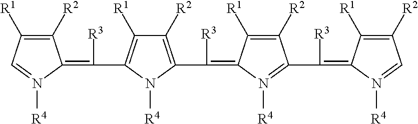

wherein each R¹ may be independently hydrogen, C₁-C₆ alkyl, C₂-C₆ alkenyl, or aldehyde; each R² may be independently hydrogen, C₁-C₆ alkyl, or aldehyde; each R³ may be independently hydrogen or C₁-C₆ alkyl; and each R⁴ may be independently absent, hydrogen, or C₁-C₆ alkyl.

In another aspect, the present technology provides a composition that may include one or more of the chlorophyll polymers described herein. The composition a may include one or more additional polymers. In some embodiments, the composition changes conductivity in the presence of a volatile compound as compared to in the absence of the volatile compound. In some embodiments, the composition changes color in the presence of a volatile compound as compared to in the absence of the volatile compound. In some embodiments, the composition may include one or more pigments. In some embodiments, the composition may be electrically conductive.

In another aspect, the present technology provides a sensor for monitoring food quality, which includes the composition. In another aspect, the present technology provides a device for packaging food, which includes the composition. In another aspect, the present technology provides a device for detecting the presence of a toxic gas, which includes the composition.

In another aspect, the present technology provides an electrically conductive material that may include one or more of the chlorophyll polymers described herein. The electrically conductive material may include one or more additional polymers. In some embodiments, the electrically conductive material changes conductivity in the presence of a volatile compound as compared to in the absence of the volatile compound. In some embodiments, the electrically conductive material changes color in the presence of a volatile compound as compared to in the absence of the volatile compound. In some embodiments, the electrically conductive material may include one or more pigments.

In another aspect, the present technology provides a sensor for monitoring food quality, which includes the electrically conductive material. In another aspect, the present technology provides a device for packaging food, which includes the electrically conductive material. In another aspect, the present technology provides a device for detecting the presence of a toxic gas, which includes the electrically conductive material.

In another aspect, the present technology provides a method of preparing a chlorophyll polymer of as described herein, which may include heating a chlorophyll in the presence of formaldehyde in a solution.

The foregoing summary is illustrative only and is not intended to be in any way limiting. In addition to the illustrative aspects, embodiments, and features described above, further aspects, embodiments, and features will become apparent by reference to the figures and the following detailed description.

### BRIEF DESCRIPTION OF THE FIGURES

FIG. 1 illustrates an exemplary reaction scheme, which represents (a) a chlorophyll molecule with Mg²⁺ in the center and a long hydrocarbon (phytol) tail; (b) removal of Mg²⁺ and the phytol tail from chlorophyll by heating in a mild formic acid solution to produce an intermediate ring tetrapyrrole with methyl side chains; (c) ring opening to produce a linear tetrapyrrole with methyl functional groups; and (d) polymerization of the linear tetrapyrroles in the presence of formaldehyde and water to produce a chlorophyll polymer with alkyl side chains.

FIGS. 2A-2F illustrate TEM images of blue and red anthocyanin nanoparticles with a polyethylene glycol (“PEG”) coating that are about 100 nm-500 nm in size. FIGS. 2A-2D show blue anthocyanin nanoparticles and FIGS. 2E-2F show red anthocyanin nanoparticles.

FIGS. 3A-3D illustrate the brightness of anthocyanin nanoparticles (FIGS. 3C and 3D) is much greater than anthocyanin macromolecules (FIGS. 3A and 3B) for both anthocyanin red and blue.

FIGS. 4A-4I illustrate chlorophyll polymers doped with red and blue anthocyanin nanoparticles and a color change of the polymers following exposure to an alkaline solution.

FIGS. 5A-5C illustrate conductivity measurements of chlorophyll polymer sensors as measured using multimeter in MΩ on three different samples of citrus fruit.

FIG. 6 illustrates an XRD of the chlorophyll polymer.

FIG. 7 illustrates an FTIR of the chlorophyll polymer.

FIGS. 8A-8C illustrate cyclic voltammetry of the chlorophyll polymer.

FIG. 9 illustrates an example of a smart package with a sensor tag including the chlorophyll polymer doped with dye nanoparticles.

### DETAILED DESCRIPTION

Chlorophyll polymers include polymers made by any of the disclosed methods. For example, in some embodiments, the chlorophyll polymer is a polymer made by a method comprising polymerizing a linear tetrapyrrole obtained from chlorophyll or a chlorophyll derivative to form a long chain chlorophyll polymer. Another embodiment includes a chlorophyll polymer made by a method comprising heating to produce a linear tetrapyrrole, and polymerizing the linear tetrapyrrole to form a long chain chlorophyll polymer.

The polymer may be derived from one or more types of chlorophyll or chlorophyll derivatives that are either naturally occurring or synthetic. “Chlorophyll derivative” refers to any compound containing a chlorin ring or other conjugated pyrrole. In one embodiment, the chlorophyll or chlorophyll derivative is subjected to conditions capable of demetallating the compound, removing one or more functional side chains, and/or opening the cyclic structure, if the compound is in the form of a macrocyclic ring (e.g., a chlorin ring). In some embodiments, the opened compound may include two or more pyrroles conjugated by an alkene. Chlorophyll polymers may be manufactured at costs as low as $1 per 100 g making chlorophyll polymer sensors or devices an attractive alternative to sensors currently on the market.

Conductive polymers refer to organic polymers that conduct electricity. These compounds may have metallic conductivity or may be semiconductors. Conductive polymers include, but are not limited to, linear-backbone “polymer blacks” (such as polyacetylene, polypyrrole, and polyaniline), and their copolymers. Some conductive polymers may have aromatic rings or double bonds in the polymer chain to provide conductivity. Nonlimiting examples of such polymers include non-heteroatom containing polymers, such as poly(fluorene)s, polyphenylenes, polypyrenes, polyazulenes, polynaphthalenes, poly(acetylene)s (PAC), and poly(p-phenylene vinylene) (PPV); nitrogen-containing polymers, such as poly(pyrrole)s (PPY), polycarbazoles, polyindoles, polyazepines, and polyanilines (PANI); and sulfur-containing polymers, such as poly(thiophene)s (PT), poly(3,4-ethylenedioxythiophene) (PEDOT), and poly(p-phenylene sulfide) (PPS). In some embodiments, the conductive polymer may be a copolymer with at least about 50 wt %, at least about 60 wt %, at least about 70 wt %, or about 80 wt %, or any range between two of the values (end points inclusive) of the conductive polymer on the surface of the copolymer. In some embodiments, the copolymer may have functionalized pores together with the nanoparticles in the material. In some embodiments, the chlorophyll polymers described herein may be conductive polymers.

In accordance with one aspect, provided are one or more chlorophyll polymers of Formula A:

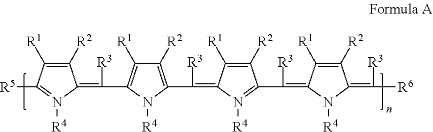

or a stereoisomer and/or tautomer thereof wherein each R¹ may be independently hydrogen, C₁-C₆ alkyl, C₂-C₆ alkenyl, or aldehyde, or one or more of R¹ can be a conductive polymer; each R² may be independently hydrogen, C₁-C₆ alkyl, or aldehyde; each R³ may be independently hydrogen, C₁-C₆ alkyl; each R⁴ may be independently absent, hydrogen or C₁-C₆ alkyl; R⁵ may be independently hydrogen or C₁-C₆ alkyl; R⁶ may be independently hydrogen or C₁-C₆ alkyl; and may be an integer of from 2 to 2000. In some embodiments, R¹ at each occurrence may be independently methyl or —CH═CH₂. In some embodiments, one or more of R¹ may be a conductive polymer. In some embodiments, one or more of R¹ may be a polypyrrole. In some embodiments, R² may be hydrogen. In some embodiments, R³ may be hydrogen. In some embodiments, R⁴ may be hydrogen. In some embodiments n may be an integer from about 10 to 1750, about 50 to 1500, or about 100 to 1000 or any range between two of the values (end points inclusive).

In some embodiments, the chlorophyll polymer may be a polymer of Formula B:

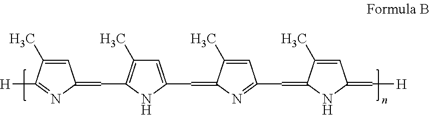

or a stereoisomer and/or tautomer thereof; wherein n may be an integer of from 2 to 2000 or as defined herein.

In another aspect, the present technology provides a compound of the formula:

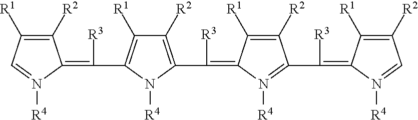

wherein each R¹ may be independently hydrogen, C₁-C₆ alkyl, C₂-C₆ alkenyl, or aldehyde; each R² may be independently hydrogen, C₁-C₆ alkyl, or aldehyde; each R³ may be independently hydrogen or C₁-C₆ alkyl; and each R⁴ may be independently absent, hydrogen or C₁-C₆ alkyl. In some embodiments, each R¹ may be independently methyl or —CH═CH₂. In some embodiments, R² may be hydrogen. In some embodiments, R³ may be hydrogen. In some embodiments, R⁴ may be hydrogen.

In another aspect, the present technology provides a chlorophyll polymer prepared by a process including heating one or more compounds of Formulas I and/or II:

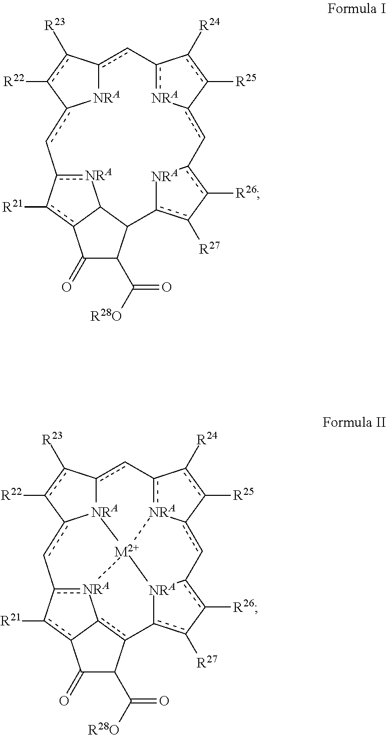

or a stereoisomer and/or tautomer thereof in a solution comprising R—CHO, wherein R may be C₁-C₄ alkyl; [symbol] represents a single bond or a double bond; R^{A} may be absent or a hydrogen; M²⁺ may be Mg²⁺, Fe²⁺, Co²⁺, Ni²⁺, or Cu²⁻; R²¹, R²², R²³, R²⁴, R²⁵, and R²⁶ at each occurrence may be independently hydrogen, C₁-C₄ alkyl, C₂-C₄ alkenyl, or aldehyde; R²⁷ may be a C₁-C₄ alkyl or C₂-C₄ alkenyl, wherein the C₁-C₄ alkyl or C₂-C₄ alkenyl may be optionally substituted with —C(O)OR²⁹, wherein R²⁹ may be hydrogen, C₁-C₂₄ alkyl, or C₂-C₂₄ alkenyl; and R²⁸ may be hydrogen or C₁-C₄ alkyl. In some embodiments, R may be methyl. In some embodiments, R²¹ may be methyl. In some embodiments, R²² may be methyl. In some embodiments, R²² may be ethyl. In some embodiments, R²³ may be methyl. In some embodiments, R²⁴ may be —CH═CH₂ or CHO. In some embodiments, R²⁵ may be methyl. In some embodiments, R²⁶ may be methyl. The solution may include formic acid, acetone, alcohol, and/or water.

In some embodiments, the compound of Formula I may be Formula III and/or the compound of Formula II may be Formula IV:

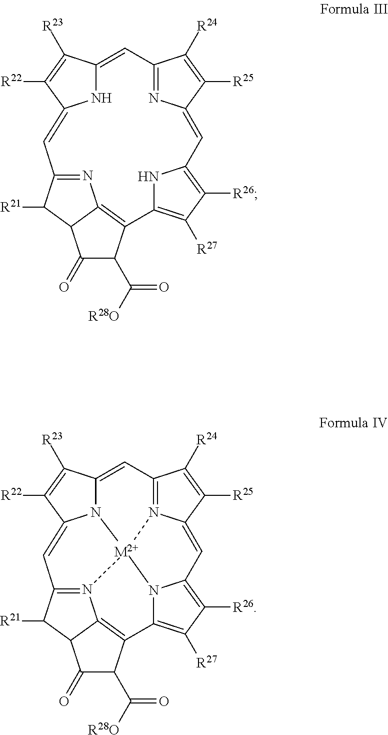

In some embodiments, M²⁺ may be Mg²⁺. In some embodiments, the compound of Formulas I and/or II maybe a chlorophyll or demetallized chlorophyll. The chlorophyll may be chlorophyll a, chlorophyll b, chlorophyll cl, chlorophyll c2, chlorophyll d, and/or chlorophyll f. In some embodiments, the chlorophyll may be in biomaterials such as plant leaves, algae, and/or cyanobacteria.

In another aspect, the present technology provides for the preparation of a chlorophyll polymer that may be the intermediate compound:

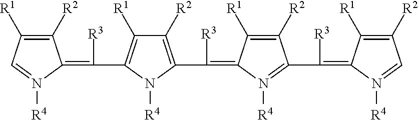

wherein each R¹ may be independently hydrogen, C₁-C₆ alkyl, C₂-C₆ alkenyl, or aldehyde; each R² may be independently hydrogen, C₁-C₆ alkyl, or aldehyde; each R³ may be independently hydrogen or C₁-C₆ alkyl; and each R⁴ may be independently absent, hydrogen or C₁-C₆ alkyl. In some aspects, each R¹ may be independently methyl or —CH═CH₂. In some aspects, R² may be hydrogen. In some aspects, R³ may be hydrogen. In some aspects, R⁴ may be hydrogen.

In another aspect, provided is a method of preparing a chlorophyll polymer described herein including heating a chlorophyll in a solution comprising aldehyde, such as an aldehyde of the formula RCHO wherein R may be C₁-C₄ alkyl, e.g., formaldehyde. In some embodiments, the aldehyde may be selected from formaldehyde (methanal), acetaldehyde (ethanal), propionaldehyde (propanal), butyraldehyde (butanal), benzaldehyde, cinnamaldehyde, vanillin, tolualdehyde, furfural, retinaldehyde, or mixtures thereof. In another aspect, the present technology provides a method of preparing a chlorophyll polymer as described herein, which may include heating a chlorophyll in the presence of formaldehyde in a solution. In some embodiments, the solution may include a solvent including an organic solvent, a protic organic solvent, an aqueous solvent, a nonprotic organic solvent, or combinations thereof. In some embodiments, the solution may include a solvent including acetone, alcohol, and/or water. In some embodiments, the chlorophyll may be in biomaterials such as plant leaves, algae, and cyanobacteria.

In another aspect, the present technology provides a method of preparing a chlorophyll polymer, which may include (a) heating a dephytylated chlorophyll or chlorophyll derivative compound where magnesium is not present to produce a linear tetrapyrrole, and (b) polymerizing the linear tetrapyrrole to form a long chain chlorophyll polymer. In another aspect, the present technology provides a chlorophyll polymer produced by the method. In another aspect, the present technology provides a method of preparing a chlorophyll polymer, which may include polymerizing a linear tetrapyrrole obtained from chlorophyll or a chlorophyll derivative to form a long chain chlorophyll polymer. In another aspect, the present technology provides a chlorophyll polymer produced by the method.

In some embodiments, the method may include adding polyvinyl alcohol to the solution to produce an elastomeric film that includes one or more of the chlorophyll polymers described herein and polyvinyl alcohol.

In some embodiments, the method may include doping the chlorophyll polymer with a pigment. The pigment may be selected from the group consisting of pentamethoxy red, hexamethoxy red, heptamethoxy red, xylenol blue (p-xylenolsulfonephthalein), phenol red, cresol red (o-cresol sulfonephthalein), neutral red (3-amino-7-dimethylamino-2-methylphenazine chloride), m-cresol purple, bromocresol purple (5′,5″-dibromo-o-cresolsulfonephthalein), bromocresol green (tetrabromo-m-cresol sulfonephthalein), crystal violet, malachite green, phenolphthalein, thymolphthalein, thymol blue, bromothymol blue (3′,3″-dibromothymolsulfonephthalein), o-cresolphthalein, p-naphtholbenzein (4-[alpha-(4-hydroxy-1-naphthyl)benzylidene]-1(4H)-naphthalenone), and combinations thereof. The dye nanoparticles may be highly pH and redox sensitive. In some embodiments, the pigment may be a pH sensitive nontoxic dye. In some aspects, the pigment may be a flower derived pigment. In some embodiments, the pigment may be a flower anthocyanin pigment. Pigments such as anthocyanin pigments may be extracted from Brassica oleracea (e.g., red cabbage). The fluorescence spectra and fluorescence excitation spectra of the pigment may be used at varying pHs.

In some embodiments, the pigment may be present as nanoparticles. In some embodiments, the size of the nanoparticles may be in a range of about 1 nm to 100 nm, about 2.5 nm to 300 nm, about 5 nm to 100 nm, or about 10 nm to 100 nm. In some embodiments, the size of the nanoparticles may be in a range of about 50 nm to 500 nm, about 75 nm to 400 nm, or about 100 nm to 300 nm. In some embodiments, the nanoparticles may be about 10 nm, 20 nm, 50 nm, 100 nm, 200 nm, 300 nm, 400 nm, or 500 nm, or within a range between any of the two numbers, end points inclusive.

In some aspects, the nanoparticles may be coated with a polymer. In some embodiments the polymer may be poly(ethylene glycol) (PEG), polyvinyl alcohol, polyethylene glycol, carboxymethyl cellulose, poly(vinylpyrrolidone), sodium dodecyl sulphate, long-chain thiol, long-chain amines, carboxylic compounds, bovine serum, albumin, cellulose, or a combination thereof. In some embodiments, the nanoparticles may be coated with polyethylene glycol. The nanoparticles may be non-toxic and safe to be doped with one or more chlorophyll polymers described herein to form a sensor. In some embodiments, the nanoparticles may be embedded in a biocompatible sensing material for fluorescence imaging such as an agarose gel.

In one aspect, the chlorophyll polymer may be elastomeric. In some embodiments, the elastomeric chlorophyll polymer may be in the form of a film.

In some aspects, the present technology provides an electrically conductive material and/or composition which include one or more of the chlorophyll polymers described above. In some embodiments, cyclic voltametry of the chlorophyll polymer or the electrically conductive material and composition that may include the chlorophyll polymer may have oxidation and reduction peaks at about 1.0 to 2.0 V and about 0.1 to 1.0 V, respectively, at 100 mV/S scanning rate. In some embodiments, the oxidation and reduction peaks may be about 1.5 to 1.6 V and about 0.5 to 0.6 V.

The electrically conductive material or composition may further include one or more additional polymers selected from the group consisting of polyvinyl alcohol, polyvinyl acetate, polyvinyl nitrate, polyvinylpyrrolidone, and polyvinylchloride. In some embodiments, the additional polymer may be polyvinyl alcohol. The electrically conductive material or composition may have a chlorophyll polymer to additional polymer ratio from about 1:10 to about 10:1, such as about 1:9, 1:8, 1:7, 1:6, 1:5, 1:4, 1:3, 1:2, 1:1, 2:1, 3:1, 4:1, 5:1, 6:1, 7:1, 8:1, 8:1, 9:1 or 10:1, or within a range between any of the two numbers, end points inclusive. The ratio of the chlorophyll polymer and the additional polymer maybe from about 1:10 to about 10:1. In some embodiments, the ratio may be about 1:1.

In some embodiments, the electrically conductive material's or composition's electric conductivity may change in the presence of a volatile compound as compared to in the absence of the volatile compound. In some embodiments, the electrically conductive material's or composition's color may change in the presence of a volatile compound as compared to in the absence of the volatile compound. In some embodiments, the amount of an increase in electric conductivity may correlate to the amount of the volatile compound as compared to in the absence of the volatile compound. In some embodiments, the volatile compound may be a volatile organic compound. In some embodiments, the volatile compound may be an acidic compound. In some embodiments, the volatile compound may be a basic compound such as an amine. The volatile compound may be selected from the group consisting of putrescine, aniline, methylamine, dimethyl amine, trimethylamine, aziridine, piperidine, polyamines, and mixtures thereof. In some embodiments, the electrically conductive material or composition may be capable of detecting a volatile compound at a concentration of about 1 ppm or higher, about 2 ppm or higher, about 3 ppm or higher, about 4 ppm or higher, about 5 ppm or higher, about 6 ppm or higher, about 7 ppm or higher, about 8 ppm or higher, about 9 ppm or higher, or about 10 ppm or higher. In some embodiments, the electrically conductive material or composition may be capable of detecting a volatile compound at a concentration of about 1 ppm or higher. In some embodiments, the electrically conductive material may be capable of detecting a volatile compound at a concentration of about 2 ppm or higher.

In some embodiments, the electrically conductive material or composition may include a pigment as described herein.

In another aspect, provided are methods for preparing a chlorophyll polymer including treating chlorophyll in the presence of an aldehyde, such as formaldehyde, and a solvent, such as water, to form an electrically conductive material.

In another aspect, the present technology provides a sensor. In one embodiment, the sensor may be a gas sensor, which includes the electrically conductive material or composition described herein. In one embodiment, the sensor may be a sensor for monitoring food quality, which includes the electrically conductive material described herein. In some embodiments, a change in conductivity of the electrically conductive material may indicate that the quality of the food may be poor. In some embodiments, a change in color of the electrically conductive material may indicate that the quality of the food may be poor. In some embodiments, the sensor may be able to rapidly detect volatile compounds from contaminated food and drink stocks. In some embodiments, the food may be a fruit, vegetables, meat, fish, or milk. In some embodiments, the sensor may be used to detect the quality of fruit. In some embodiments, the sensor may be able to identify average or poor quality fruit from high quality fruit. The sensor may be used to differentiate between high quality fruit and average or low quality fruit by detecting the VOC and/or moisture differences released from the fruit. In some embodiments, the electrically conductive material or composition may indicate high conductivity when used to test a high quality fruit compared to an average quality fruit. For example, the electrically conductive material or composition may indicate high conductivity when testing a high quality mango (i.e. sweet aroma) compared to an average mango. Fruit may include, but is not limited to, mango, citrus fruit (e.g., lime), banana, and grapes.

In some aspects, the sensor may be incorporated into food packaging. The sensors may be able to perform odor detection continually. In some embodiments, the sensor may be integrated into a single chip for further signal processing, which may lead to sensor arrays. In some embodiments, the chlorophyll polymer included in the sensor may be doped with other doping agents to make various types of sensor arrays for detection of odor, taste, color, and/or other biomolecules in beverages and foods including those not in packaging and those in processed packaging.

In some embodiments, the sensor may be reusable after being reset to a pre-detection state. In some embodiments, the sensor may be reset by removing volatile compounds from the area surrounding the sensor and/or by heating the sensor for a short period of time (e.g., less than about 60 seconds, less than about 45 seconds, or less than about 30 seconds) at about 50-100° C., about 55-90° C., or about 60-80° C. In some embodiments, the sensor may be reset by heating the sensor for less than about 60 seconds at about 60-80° C. The sensors may be used 1000 or more times. The sensors may be used 500 or more times. The sensors may be used 250 or more times.

In another aspect the present technology provides a method for making sensors to determine beverage and food quality.

In another aspect, the present technology provides a device for packaging food, which includes the electrically conductive material or composition described herein. In some embodiments, a change in conductivity of the electrically conductive material or composition may indicate that the quality of the food may be poor. In some embodiments, a change in color of the electrically conductive material or composition may indicate that the quality of the food may be poor. In some embodiments, the food may be a fruit, vegetables, meat, fish, or milk. In some embodiments, the device may be used to detect the quality of fruit.

In another aspect, the present technology provides a device for detecting the presence of a toxic gas that includes one or more of the chlorophyll polymers. In another aspect, the present technology provides a device for detecting the presence of a toxic gas that includes the electrically conductive material or composition described herein. In some embodiments, a change in conductivity of the electrically conductive material or composition may indicate the presence of the toxic gas. In some embodiments, a change in color of the electrically conductive material or composition may indicate the presence of the toxic gas. In some embodiments, the toxic gas may be selected from the group consisting of carbon monoxide, methane, acetylene, ammonia, boron tribromide, chlorine, methyl chloride, methyl bromide, methyl isocyanate, methyl mercaptan, nitric oxide, phosphine, and mixtures thereof.

### EXAMPLES

The following examples are intended to more specifically illustrate the present cleaning compositions according to various embodiments described above. These examples should in no way be construed as limiting the scope of the present technology.

### Example 1

### Synthesis of a Chlorophyll Polymer

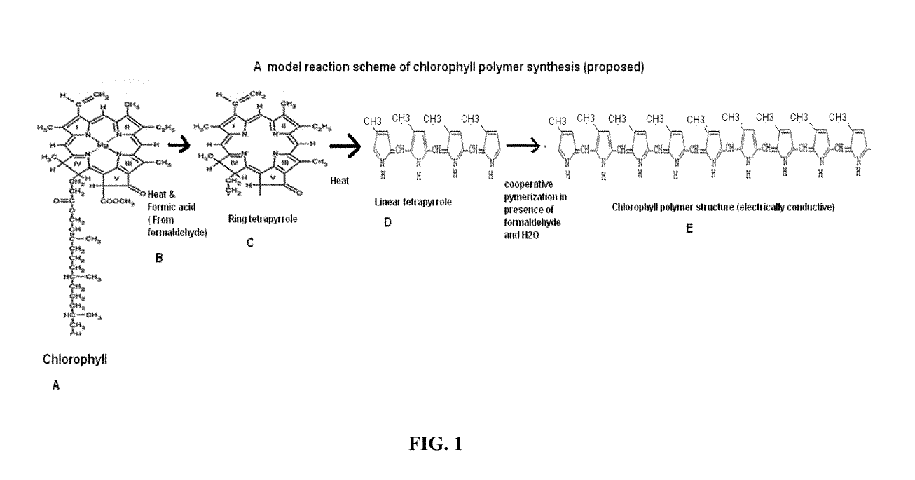

An exemplary reaction scheme is shown in FIG. 1. Plant leaves were cut in to small pieces and ground by mortar and pestle followed by the addition of acetone to extract chlorophyll. After 30 minutes the acetone with extracted chlorophyll was filtered and poured in a 250 ml conical flask.

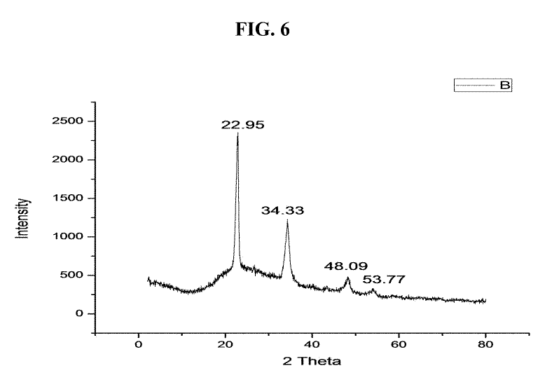

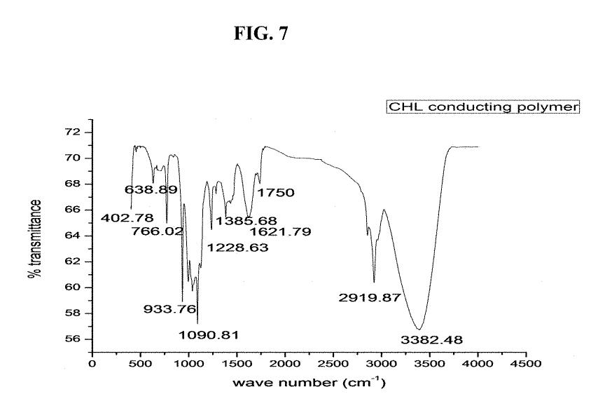

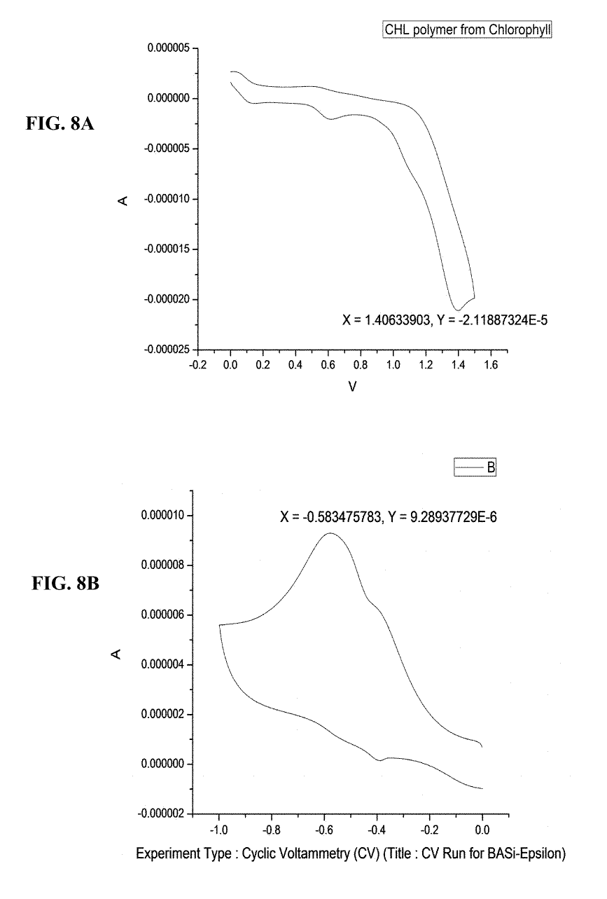

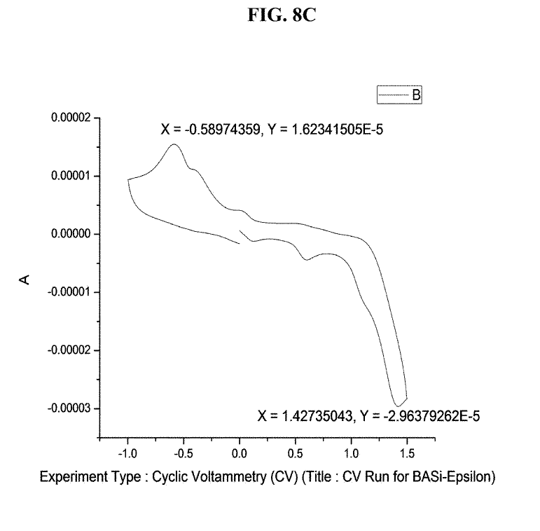

The extracted chlorophyll was mixed with formaldehyde and subjected to heat to remove the Mg²⁺ and the phytol tail (FIG. 1B) to produce a cyclic tetrapyrrole (FIG. 1C). Removal of Mg²⁺ and the phytol tail was followed by heating in the presence of formaldehyde, acetone, and water to open the ring and produce linear a tetrapyrrole (FIG. 1D). The linear tetrapyrrole tetramers were subjected to polymerization in a solution with polyvinyl alcohol, formaldehyde, and water through ionic propagation to yield high molecular weight chlorophyll polymers. The polymer product was a thin conductive film with elastomeric properties. The polymer was characterized using XRD (FIG. 6), FTIR (FIG. 7), and cyclic voltammetry (FIGS. 8A-8C). The chlorophyll polymer had an oxidation peak at 1.41 V (FIG. 8A) and a reduction peak at 0.58 V (FIG. 8B). After dissolving the chlorophyll polymer in acetonitrile, the solution had an oxidation peak of 1.42V and a reduction peak of 0.59 V (FIG. 8C).

### Example 2

### the Chlorophyll Polymer as Gas Sensor

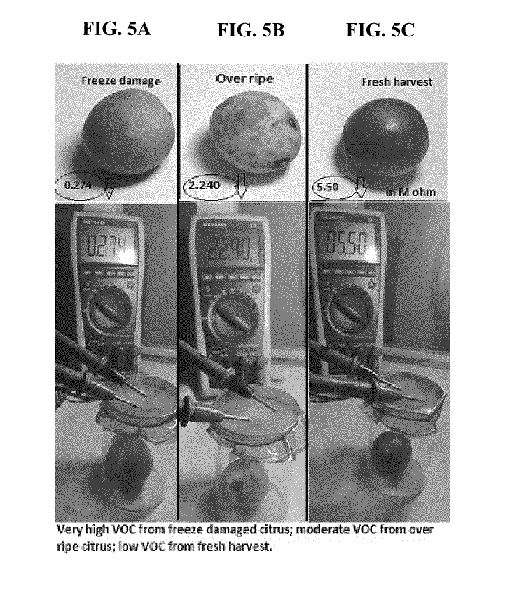

Freeze damaged, over ripe, and freshly harvested limes were placed inside three different beakers that were tightly covered by the chlorophyll polymer using a rubber band. The beakers were allowed to sit covered for 15 minutes. The electrical resistance of the chlorophyll polymers was measured. The freeze damaged lime (FIG. 5A) and over ripe lime (FIG. 5B) showed reduced resistance/increased conductivity (i.e., high VOC). In contrast, the fresh lime showed high resistance/low conductivity (i.e, low VOC) (FIG. 5C).

Over ripe and freshly harvested bananas were placed inside two different beakers and tightly covered by the chlorophyll polymer using a rubber band. The beakers were allowed to sit covered for 15 minutes. The over ripe banana showed very low resistance (0.082 MΩ) compared to the freshly harvested banana (4.21 MΩ) indicating higher VOC release from the over ripe banana.

Over ripe and freshly harvested grapes were placed inside two different beakers and tightly covered by the chlorophyll polymer using a rubber band. The beakers were allowed to sit covered for 15 minutes. The over ripe grapes showed very low resistance (0.186 MΩ) compared to the freshly harvested grapes (2.740 MΩ) indicating higher VOC release from the over ripe grapes.

### Example 3

### Doping the Chlorophyll Polymer with a Pigment Nanoparticle

Anthocyanin pigment was extracted from blue and red flowers using alcohol/water solvent extraction. The extracts were then filtered through filter paper and stored inside a refrigerator.

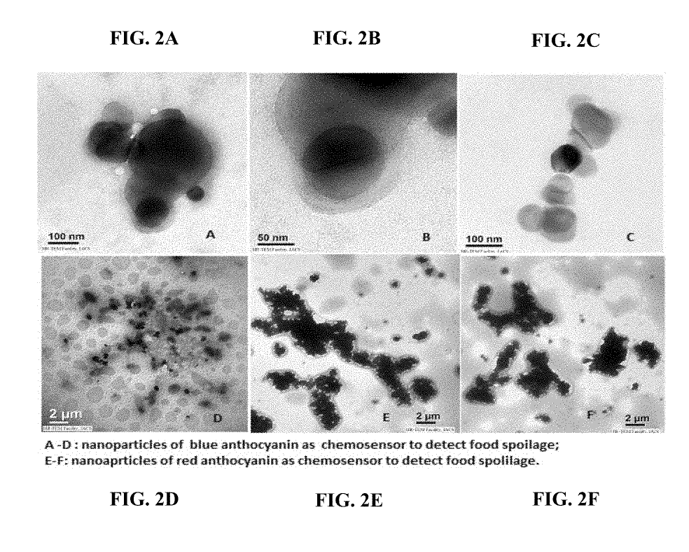

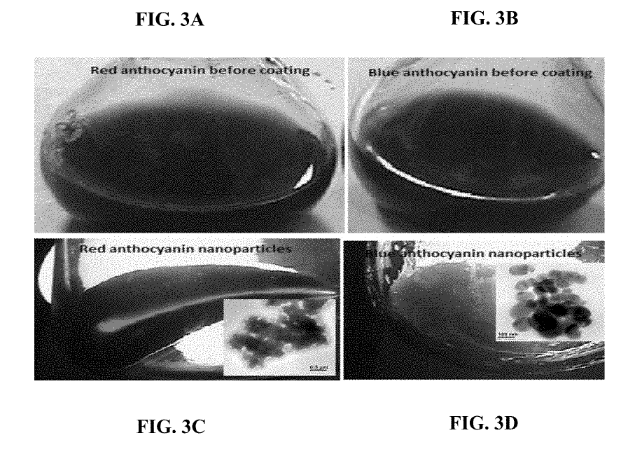

The aqueous pigment extracts were then stirred vigorously on a magnetic stirrer while a 20% aqueous solution of polyethylene glycol (“PEG”) 400/acacia gum solution was added drop-wise continuously. The dye nanoparticle solution are characterized by TEM (FIGS. 2A-2F). The PEG coated blue and red anthocyanin nanoparticles were in the range of 100 nm to 500 nm. FIGS. 3A-3D show the PEG coated anthocyanin nanoparticles are brighter than anthocyanin macromolecules.

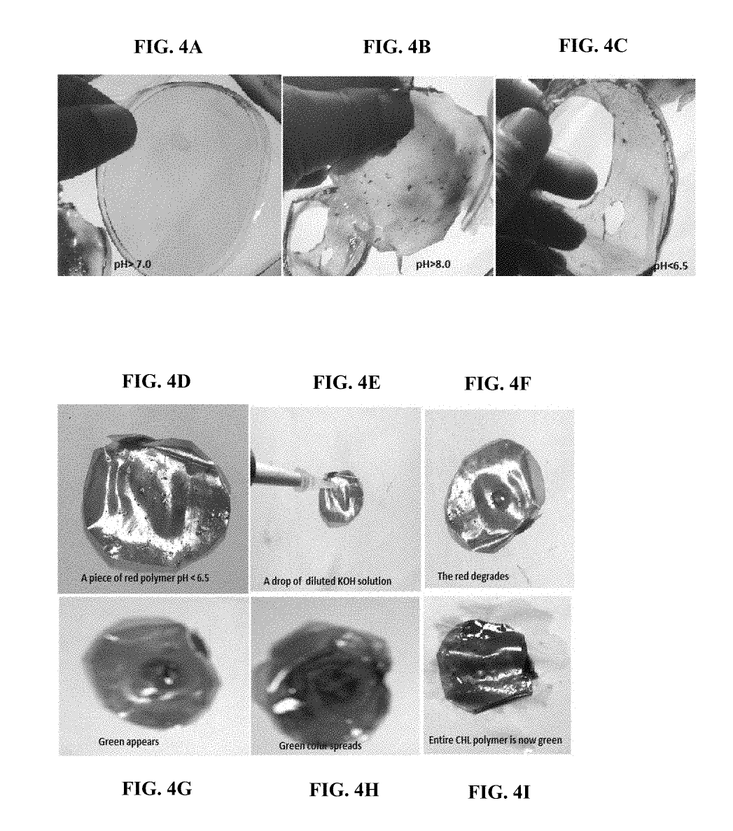

The anthocyanin nanoparticles were then poured on freshly prepared chlorophyll polymers in petriplates. The pigment nanoparticle doped polymers were then allowed to dry in a hot air oven for 2 hours before being removed from the petriplate. The polymers doped with blue anthocyanin nanoparticles formed yellow and green chlorophyll polymers (FIGS. 4A-4B) and the polymers doped with red anthocyanin nanoparticles formed red chlorophyll polymers (FIG. 4C). The red chlorophyll polymer gradually turned green when exposed to an alkaline solution (FIGS. 4D-4I).

### Example 4

### Dye Nanoparticle Doped Chlorophyll Polymer as a Color Sensor/Device

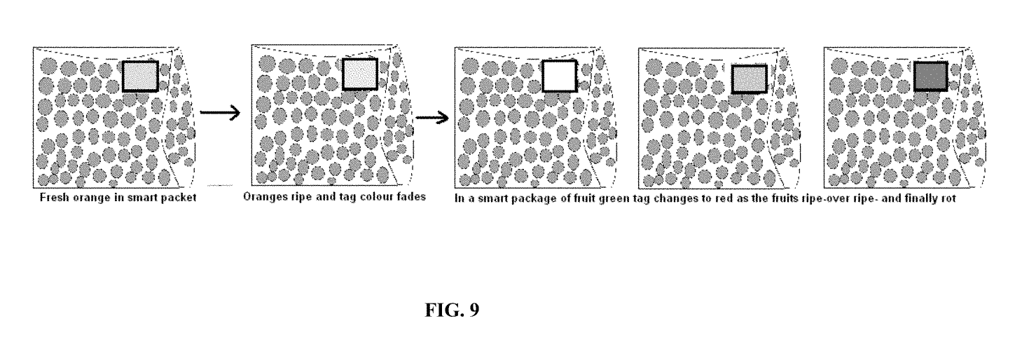

The green dye nanoparticle doped chlorophyll polymer was deposited on a small area of a packaging film that was exposed to the packaged food (e.g., orange) (FIG. 9). The polymer gradually turned from green to red indicating the increased presence of VOCs being released from the over ripe orange.

The following terms are used herein, the definitions of which are provided for guidance.

For the purposes of this disclosure and unless otherwise specified, “a” or “an” means “one or more.”

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% or up to plus or minus 5% of the stated value.

“Alkyl” refers to monovalent straight or branched saturated aliphatic hydrocarbyl groups. In some embodiments, an alkyl has from 1 to 20 carbon atoms, from 1 to 10 carbon atoms or from 1 to 6 carbon atoms. This term includes, by way of example, linear and branched hydrocarbyl groups such as methyl (CH₃—, or Me), ethyl (CH₃CH₂—, or Et), n-propyl (CH₃CH₂CH₂—, or n-Pr), isopropyl ((CH₃)₂CH—, or i-Pr), n-butyl (CH₃CH₂CH₂CH₂—, or n-Bu), isobutyl ((CH₃)₂CHCH₂—, or i-Bu), sec-butyl ((CH₃)(CH₃CH₂)CH—, or s-Bu), t-butyl ((CH₃)₃C—, or t-Bu), n-pentyl (CH₃CH₂CH₂CH₂CH₂—), and neopentyl ((CH₃)₃CCH₂—). Cₓ alkyl refers to an alkyl group having x number of carbon atoms.

“Alkenyl” refers to monovalent straight or branched hydrocarbyl groups having at least 1, such as 1 or 2 sites, of vinyl (>C═C<) unsaturation. In some embodiments, an alkenyl has from 2 to 10 carbon atoms, 2 to 6 carbon atoms or from 2 to 4 carbon atoms. Such groups are exemplified, for example, by vinyl, allyl, and but-3-en-1-yl. Included within this term are the cis and trans isomers or mixtures of these isomers. Cₓ alkenyl refers to an alkenyl group having x number of carbon atoms.

“Hydrogen” refers —H.

“Aldehyde” refers to —CH₂O or alkyl or alkenyl substituted with CHO, wherein CHO represents

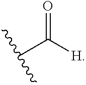

“Stereoisomer” or “stereoisomers” refer to compounds that have the same molecular formula and sequence of bonded atoms, but that differ only in the three-dimensional orientations of their atoms in space, such as compounds differ in the chirality of one or more stereocenters. Stereoisomers include cis-isomers, trans-isomers, enantiomers and diastereomers. Unless specified, a stereoisomer refers to a single stereoisomer, such as a enantiomerically pure compound or a mixture of two or more stereoisomers, such as a racemic mixture.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Active packaging,” “intelligent packaging,” and “smart packaging” refer to packaging systems used with foods, pharmaceuticals, and other types of products, which help extend shelf life, monitor freshness, display information on quality, improve safety, and improve convenience.

“Chlorophyll polymer” or “a chlorophyll polymer” refers to one or more polymers described herein. Such chlorophyll polymers may be made by any of the methods described above including polymerizing a linear tetrapyrrole obtained from chlorophyll or a chlorophyll derivative to form a long chain polymer.

“Poor” food quality refers to food including, but not limited to, fruits, vegetables, meats, fish, and milk that is over-ripened, spoiled, rotten, decomposing, and/or not edible.

### Illustrative Embodiments

Reference is made in the following to a number of illustrative embodiments of the subject matter described herein. The following embodiments describe illustrative embodiments that may include various features, characteristics, and advantages of the subject matter as presently described. Accordingly, the following embodiments should not be considered as being comprehensive of all of the possible embodiments or otherwise limit the scope of the methods, materials and compositions described herein.

In one aspect, the present technology provides a chlorophyll polymer of Formula A:

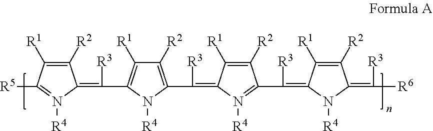

wherein the chlorophyll polymer may be derived from chlorophyll; R¹ at each occurrence may be independently hydrogen, C₁-C₆ alkyl, C₂-C₆ alkenyl, aldehyde, or a conductive polymer; R² at each occurrence may be independently hydrogen, C₁-C₆ alkyl, or aldehyde; R³ at each occurrence may be independently hydrogen or C₁-C₆ alkyl; R⁴ at each occurrence may be independently absent, hydrogen, or C₁-C₆ alkyl; R⁵ at each occurrence may be independently hydrogen or C₁-C₆ alkyl; R⁶ at each occurrence may be independently hydrogen or C₁-C₆ alkyl; and n may be an integer of from 2 to 2000. In some embodiments, R¹ at each occurrence may be independently methyl or —CH═CH₂. In some embodiments, one or more of R¹ may be a conductive polymer. In some embodiments, one or more of R¹ may be a polypyrrole. In some embodiments, R² may be hydrogen. In some embodiments, R³ may be hydrogen. In some embodiments, R⁴ may be hydrogen. In some embodiments, the chlorophyll polymer may be a polymer of Formula B:

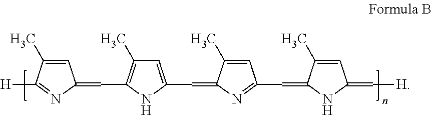

In another aspect, the present technology provides a chlorophyll polymer prepared by a process comprising heating a compound of Formulas I and/or II:

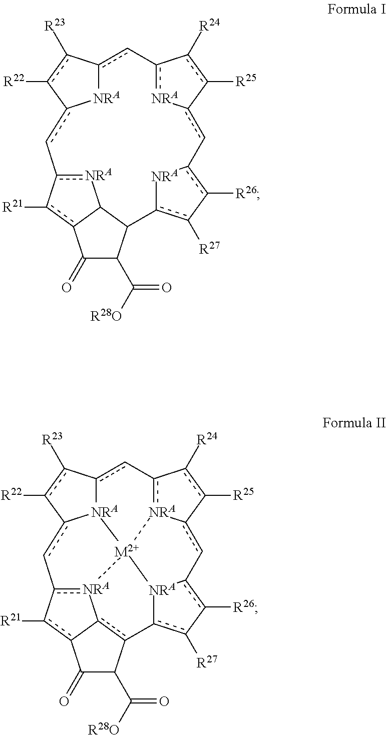

or a stereoisomer and/or tautomer thereof in a solution comprising R—CHO, wherein R may be C₁-C₄ alkyl; [symbol] represents a single bond or a double bond; R^{A} may be absent or a hydrogen; M²⁺ may be Mg²⁺, Fe²⁺, Co²⁺, Ni²⁺, or Cu²⁻; R²¹, R²², R²³, R²⁴, R²⁵, and R²⁶ at each occurrence may be independently hydrogen, C₁-C₄ alkyl, C₂-C₄ alkenyl, or aldehyde; R²⁷ may be a C₁-C₄ alkyl or C₂-C₄ alkenyl, wherein the C₁-C₄ alkyl or C₂-C₄ alkenyl may be optionally substituted with —C(O)OR²⁹, wherein R²⁹ may be hydrogen, C₁-C₂₄ alkyl, or C₂-C₂₄ alkenyl; and R²⁸ may be hydrogen or C₁-C₄ alkyl. The solution may include formic acid, acetone, alcohol, and/or water. In some embodiments, M²⁺ may be Mg²⁺. In some embodiments, the compound of Formula I may be Formula III and/or the compound of Formula II may be Formula IV:

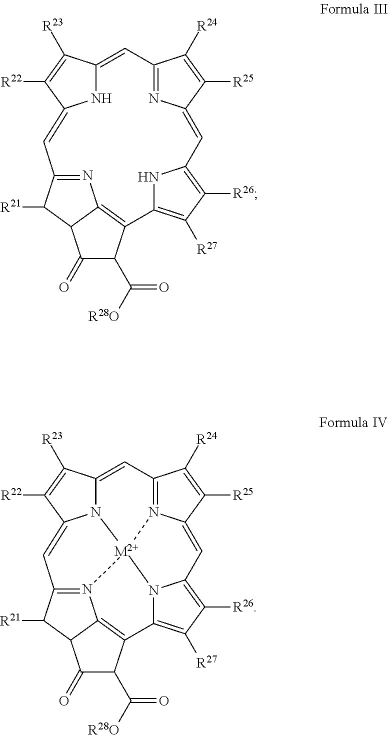

In some embodiments, the compound of Formulas I and/or II may be a chlorophyll. The chlorophyll may be chlorophyll a, chlorophyll b, chlorophyll c1, chlorophyll c2, chlorophyll d, and/or chlorophyll f In some embodiments, R may be methyl.

In another aspect, the present technology provides an electrically conductive material any one or more of the chlorophyll polymers described above. The electrically conductive material may further include one or more additional polymers selected from the group consisting of polyvinyl alcohol, polyvinyl acetate, polyvinyl nitrate, polyvinylpyrrolidone, and polyvinylchloride. In some embodiments, the additional polymer may be polyvinyl alcohol. The ratio of the chlorophyll polymer and the additional polymer maybe from about 1:10 to about 10:1. In some embodiments, the ratio may be about 1:1.

In some embodiments, the electrically conductive material's electric conductivity may change in the presence of a volatile compound as compared to in the absence of the volatile compound. In some embodiments, the electrically conductive material's color may change in the presence of a volatile compound as compared to in the absence of the volatile compound. In some embodiments, the amount of an increase in electric conductivity may correlate to the amount of the volatile compound as compared to in the absence of the volatile compound. In some embodiments, the volatile compound may be a volatile organic compound. In some embodiments, the volatile compound may be an acidic compound. The volatile compound may be selected from the group consisting of putrescine, aniline, methylamine, dimethylamine, trimethylamine, aziridine, piperidine, polyamines, and mixtures thereof. In some embodiments, the electrically conductive material may be capable of detecting a volatile compound at a concentration of about 1 ppm or higher.

In some embodiments, the electrically conductive material may include a pigment. The pigment may be selected from the group consisting of pentamethoxy red, hexamethoxy red, heptamethoxy red, xylenol blue (p-xylenolsulfonephthalein), phenol red, cresol red (o-cresol sulfonephthalein), neutral red (3-amino-7-dimethylamino-2-methylphenazine chloride), m-cresol purple, bromocresol purple (5′,5″-dibromo-o-cresolsulfonephthalein), bromocresol green (tetrabromo-m-cresol sulfonephthalein), crystal violet, malachite green, phenolphthalein, thymolphthalein, thymol blue, bromothymol blue (3′,3″-dibromothymol sulfonephthalein), o-cresolphthalein, p-naphtholbenzein (4-[alpha-(4-hydroxy-1-naphthyl)benzylidene]-1(4H)-naphthalenone), and combinations thereof. In some embodiments, the pigment may be a pH sensitive nontoxic dye. In some embodiments, the pigment may be a flower anthocyanin pigment.

In some embodiments, the pigment may be present as nanoparticles. The size of the nanoparticles may be in a range of about 100 nm to about 500 nm. In some embodiments, the nanoparticles may be coated with a polymer. In some embodiments, the nanoparticles may be coated with polyethylene glycol.

In another aspect, the present technology provides a sensor for monitoring food quality, which includes the electrically conductive material described above. In some embodiments, a change in conductivity of the electrically conductive material may indicate that the quality of the food may be poor. In some embodiments, a change in color of the electrically conductive material may indicate that the quality of the food may be poor. In some embodiments, the food may be a fruit, meat, fish, or milk.

In another aspect, the present technology provides a device for packaging food, which includes the electrically conductive material described above. In some embodiments, a change in conductivity of the electrically conductive material may indicate that the quality of the food may be poor. In some embodiments, a change in color of the electrically conductive material may indicate that the quality of the food may be poor. In some embodiments, the food may be a fruit, meat, fish, or milk.

In another aspect, the present technology provides a device for detecting the presence of a toxic gas comprising the electrically conductive material described above. In some embodiments, a change in conductivity of the electrically conductive material may indicate the presence of the toxic gas. In some embodiments, a change in color of the electrically conductive material may indicate the presence of the toxic gas. In some embodiments, the toxic gas may be selected from the group consisting of carbon monoxide, methane, acetylene, ammonia, boron tribromide, chlorine, methyl chloride, methyl bromide, methyl isocyanate, methyl mercaptan, nitric oxide, phosphine, and mixtures thereof.

In another aspect, the present technology provides a method of preparing a chlorophyll polymer of as described above, which may include heating a chlorophyll in the presence of formaldehyde in a solution. In some embodiments, the solution may include a solvent comprising acetone, alcohol and/or water. The method may include adding polyvinyl alcohol to the solution to produce an elastomeric film comprising a chlorophyll polymer and polyvinyl alcohol.

In another aspect, the present technology provides a compound of the formula:

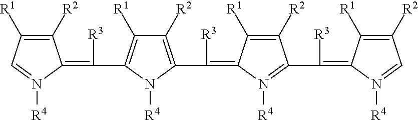

wherein each R¹ may be independently hydrogen, C₁-C₆ alkyl, C₂-C₆ alkenyl, or aldehyde; each R² may be independently hydrogen, C₁-C₆ alkyl, or aldehyde; each R³ may be independently hydrogen or C₁-C₆ alkyl; and each R⁴ may be independently absent, hydrogen or C₁-C₆ alkyl. In some embodiments, each R¹ may be independently methyl or —CH═CH₂. In some embodiments, R² may be hydrogen. In some embodiments, R³ may be hydrogen. In some embodiments, R⁴ may be hydrogen.

In another aspect, the present technology provides a composition, which may include an electrically conductive chlorophyll polymer. The composition may further include one or more additional polymers selected from the group consisting of polyvinyl alcohol, polyvinyl acetate, polyvinyl nitrate, polyvinylpyrrolidone, and polyvinylchloride. In some embodiments, the additional polymer may be polyvinyl alcohol. In another aspect, the present technology provides a composition, which may include an electrically conductive chlorophyll polymer doped with polyvinyl alcohol. The ratio of the chlorophyll polymer and the additional polymer maybe from about 1:10 to about 10:1. In some embodiments, the ratio may be about 1:1.

In some embodiments, the composition's electric conductivity may change in the presence of a volatile compound as compared to in the absence of the volatile compound. In some embodiments, the composition's color may change in the presence of a volatile compound as compared to in the absence of the volatile compound. In some embodiments, the amount of an increase in electric conductivity may correlate to the amount of the volatile compound as compared to in the absence of the volatile compound. In some embodiments, the volatile compound may be a volatile organic compound. In some embodiments, the volatile compound may be an acidic compound. The volatile compound may be selected from the group consisting of putrescine, aniline, methylamine, dimethylamine, trimethylamine, aziridine, piperidine, polyamines, and mixtures thereof. In some embodiments, the composition may be capable of detecting a volatile compound at a concentration of about 1 ppm or higher.

In some embodiments, the composition may include a pigment. The pigment may be selected from the group consisting of pentamethoxy red, hexamethoxy red, heptamethoxy red, xylenol blue (p-xylenolsulfonephthalein), phenol red, cresol red (o-cresolsulfonephthalein), neutral red (3-amino-7-dimethylamino-2-methylphenazine chloride), m-cresol purple, bromocresol purple (5′,5″-dibromo-o-cresolsulfonephthalein), bromocresol green (tetrabromo-m-cresol sulfonephthalein), crystal violet, malachite green, phenolphthalein, thymolphthalein, thymol blue, bromothymol blue (3′,3″-dibromothymolsulfonephthalein), o-cresolphthalein, p-naphtholbenzein (4-[alpha-(4-hydroxy-1-naphthyl)benzylidene]-1(4H)-naphthalenone), and combinations thereof. In some embodiments, the pigment may be a pH sensitive nontoxic dye. In some embodiments, the pigment may be a flower anthocyanin pigment.

In some embodiments, the pigment may be present as nanoparticles. The size of the nanoparticles may be in a range of about 100 nm to about 500 nm. In some embodiments, the nanoparticles may be coated with a polymer. In some embodiments, the nanoparticles may be coated with polyethylene glycol.

In another aspect, the present technology provides a method of preparing a chlorophyll polymer, which may include (a) heating a dephytylated chlorophyll or chlorophyll derivative compound where magnesium is not present to produce a linear tetrapyrrole, and (b) polymerizing the linear tetrapyrrole to form a long chain chlorophyll polymer. In another aspect, the present technology provides a chlorophyll polymer produced by the method. In another aspect, the present technology provides a method of preparing a chlorophyll polymer, which may include polymerizing a linear tetrapyrrole obtained from chlorophyll or a chlorophyll derivative to form a long chain chlorophyll polymer. In another aspect, the present technology provides a chlorophyll polymer produced by the method.

In another aspect, the present technology provides a device for detecting the presence of a toxic gas that includes one or more of the chlorophyll polymers. In another aspect, the present technology provides an electrically conductive material that includes one or more of the chlorophyll polymers.

In some embodiments, the electrically conductive material may include one or more additional polymers selected from polyvinyl alcohol, polyvinyl acetate, polyvinyl nitrate, polyvinylpyrrrolidone, and polyvinylchloride. In some embodiments, the additional polymer may be polyvinyl alcohol. In some embodiments, the ratio of the chlorophyll polymer and the additional polymer may be from about 1:10 to about 10:1. In some embodiments, the ratio of the chlorophyll polymer and the additional polymer may be from about 1:1.

In some embodiments, the electric conductivity of the electrically conductive material may change in the presence of a volatile compound as compared to in the absence of the volatile compound. In some embodiments, the color of the electrically conductive material may change in the presence of a volatile compound as compared to in the absence of the volatile compound. In some embodiments, the amount of an increase in electric conductivity may correlate to the amount of the volatile compound as compared to in the absence of the volatile compound. The volatile compound may be a volatile organic compound. The volatile compound may be an acidic compound. The volatile compound may be selected from the group consisting of putrescine, aniline, methylamine, dimethylamine, trimethylamine, aziridine, piperidine, polyamines, and mixtures thereof. In some embodiments, the electrically conductive material may be capable of detecting a volatile compound at a concentration of about 1 ppm or higher.

In some embodiments, the electrically conductive material may include a pigment. The pigment may be selected from the group consisting of pentamethoxy red, hexamethoxy red, heptamethoxy red, xylenol blue (p-xylenolsulfonephthalein), phenol red, cresol red (o-cresol sulfonephthalein), neutral red (3-amino-7-dimethylamino-2-methylphenazine chloride), m-cresol purple, bromocresol purple (5′,5″-dibromo-o-cresolsulfonephthalein), bromocresol green (tetrabromo-m-cresol sulfonephthalein), crystal violet, malachite green, phenolphthalein, thymolphthalein, thymol blue, bromothymol blue (3′,3″-dibromothymolsulfonephthalein), o-cresolphthalein, p-naphtholbenzein (4-[alpha-(4-hydroxy-1-naphthyl)benzylidene]-1(4H)-naphthalenone), and combinations thereof. In some embodiments, the pigment may be a pH sensitive nontoxic dye. In some embodiments, the pigment may be a flower anthocyanin pigment. In some embodiments, the pigment may be present as nanoparticles. The size of the nanoparticles may be in a range of about 100 nm to about 500 nm. In some embodiments, the nanoparticles may be coated with a polymer. In some embodiments, the nanoparticles may be coated with polyethylene glycol.

In another aspect, the present technology provides a sensor for monitoring food quality, which may include the electrically conductive material. In some embodiments, a change in conductivity of the electrically conductive material in the sensor may indicate that the quality of the food may be poor. In some embodiments, a change in color of the electrically conductive material in the sensor may indicate that the quality of the food may be poor. In some embodiments, the food may be a fruit, meat, fish, or milk.

In another aspect, the present technology provides a device for packaging food, which may include the electrically conductive material. In some embodiments, a change in conductivity of the electrically conductive material in the device may indicate that the quality of the food may be poor. In some embodiments, a change in color of the electrically conductive material in the device may indicate that the quality of the food may be poor. In some embodiments, the food may be a fruit, meat, fish, or milk.

In another aspect, the present technology provides a device for detecting the presence of a toxic gas, which may include the electrically conductive material. In some embodiments, a change in conductivity of the electrically conductive material in the device may indicate the presence of the toxic gas. In some embodiments, a change in color of the electrically conductive material in the device may indicate the presence of the toxic gas. The toxic gas may be selected from the group consisting of CO, methane, acetylene, ammonia, boron tribromide, chlorine, methyl chloride, methyl bromide, methyl isocyanate, methyl mercaptan, nitric oxide, phosphine, and mixtures thereof.

While certain embodiments have been illustrated and described, it should be understood that changes and modifications can be made therein in accordance with ordinary skill in the art without departing from the technology in its broader aspects.

The embodiments, illustratively described herein may suitably be practiced in the absence of any element or elements, limitation or limitations, not specifically disclosed herein. Thus, for example, the terms “comprising,” “including,” “containing,” shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof.

1. A chlorophyll polymer of Formula A: 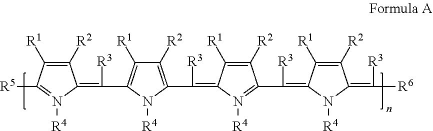
wherein:
the chlorophyll polymer is derived from chlorophyll;
R¹ at each occurrence is independently hydrogen, C₁-C₆ alkyl, C₂-C₆ alkenyl, aldehyde, or a conductive polymer;
R² at each occurrence is independently hydrogen, C₁-C₆ alkyl, or aldehyde;
R³ at each occurrence is independently hydrogen or C₁-C₆ alkyl;
R⁴ at each occurrence is independently absent, hydrogen, or C₁-C₆ alkyl;
R⁵ at each occurrence is independently hydrogen or C₁-C₆ alkyl;
R⁶ at each occurrence is independently hydrogen or C₁-C₆ alkyl; and
n is an integer of from 2 to 2000.

2. The chlorophyll polymer of claim 1, wherein R¹ at each occurrence is independently methyl or —CH═CH₂.

3. (canceled)

4. The chlorophyll polymer of claim 1, wherein one or more of R¹ is a polypyrrole.

5. The chlorophyll polymer of claim 1, wherein R² is hydrogen.

6. The chlorophyll polymer of claim 1, wherein R³ is hydrogen.

7. The chlorophyll polymer of claim 1, wherein R⁴ is hydrogen.

8. The chlorophyll polymer of claim 1, wherein the polymer is Formula B:

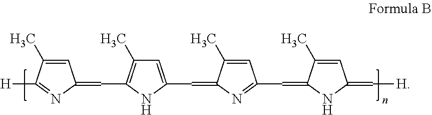

9-16. (canceled)

17. An electrically conductive material comprising a chlorophyll polymer of Formula A: 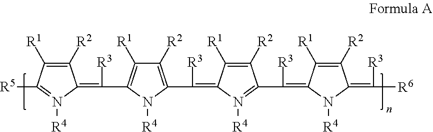
wherein:
R¹ at each occurrence is independently hydrogen, C₁-C₆ alkyl, C₂-C₆ alkenyl, aldehyde, or a conductive polymer;
R² at each occurrence is independently hydrogen, C₁-C₆ alkyl, or aldehyde;
R³ at each occurrence is independently hydrogen or C₁-C₆ alkyl;
R⁴ at each occurrence is independently absent, hydrogen, or C₁-C₆ alkyl;
R⁵ at each occurrence is independently hydrogen or C₁-C₆ alkyl;
R⁶ at each occurrence is independently hydrogen or C₁-C₆ alkyl; and
n is an integer of from 2 to 2000.

18. The electrically conductive material of claim 17, further comprising one or more additional polymers selected from the group consisting of polyvinyl alcohol, polyvinyl acetate, polyvinyl nitrate, polyvinylpyrrolidone, and polyvinylchloride.

19. (canceled)

20. The electrically conductive material of claim 18, wherein the ratio of the chlorophyll polymer and the additional polymer is from about 1:10 to about 10:1.

21. (canceled)

22. The electrically conductive material of claim 17, wherein the electric conductivity of the material increases and directly correlates to the amount of a volatile compound present as compared to in the absence of the volatile compound.

23. (canceled)

24. The electrically conductive material of claim 22, wherein the volatile compound is a volatile organic compound.

25. (canceled)

26. The electrically conductive material of claim 22, wherein the volatile compound is selected from the group consisting of putrescine, aniline, methyl amine, dimethylamine, trimethylamine, aziridine, piperidine, polyamines, and mixtures thereof.

27. The electrically conductive material of claim 17, further comprising a pigment.

28. The electrically conductive material of claim 27, wherein the pigment is selected from the group consisting of pentamethoxy red, hexamethoxy red, heptamethoxy red, xylenol blue (p-xylenolsulfonephthalein), phenol red, cresol red (o-cresol sulfonephthalein), neutral red (3-amino-7-dimethylamino-2-methylphenazine chloride), m-cresol purple, bromocresol purple (5′,5″-dibromo-o-cresolsulfonephthalein), bromocresol green (tetrabromo-m-cresol sulfonephthalein), crystal violet, malachite green, phenolphthalein, thymolphthalein, thymol blue, bromothymol blue (3′,3″-dibromothymol sulfonephthalein), o-cresolphthalein, p-naphtholbenzein (4-[alpha-(4-hydroxy-1-naphthyl)benzylidene]-1(4H)-naphthalenone), and combinations thereof.

29-30. (canceled)

31. The electrically conductive material of claim 27, wherein the pigment is present as nanoparticles.

32. (canceled)

33. The electrically conductive material of claim 31, wherein the nanoparticles are coated with a polymer.

34-35. (canceled)

36. A sensor for monitoring food quality comprising the electrically conductive material of claim 17, wherein a change in conductivity or a change in color of the electrically conductive material indicates that quality of food is poor.

35-39. (canceled)

40. A device for packaging food comprising the electrically conductive material of claim 17, wherein a change in conductivity or a change in color of the electrically conductive material indicates that quality of food is poor.

41-43. (canceled)

44. A device for detecting the presence of a toxic gas comprising the electrically conductive material of claim 17, wherein a change in conductivity or a change in color of the electrically conductive material indicates that quality of food is poor.

45-91. (canceled)
